(19) United States
(12) Patent Application Publication
(10) Pub. No.: US 2010/0203101 A1
(43) Pub. Date: Aug. 12, 2010

(54) BIO-MEMBRANE FOR TISSUE REGENERATION

(75) Inventors: Ranieri CANCEDDA, (IT); Maddalena MASTROGIACOMO, GENOVA (IT); Marco SCALA, GENOVA (IT)

Correspondence Address: Kenneth;SHARPLES, LAW OFFICE OF KENNETH K SHARPLES, SENA PLAZA, SUITE 54, 125 EAST PALACE AVE, SANTA FE, NM, 87501, US

(73) Assignee: BIORIGEN S.R.L., GENOVA (IT)

(21) Appl. No.: 12/301,881

(22) PCT Filed: May 31, 2007
(86) PCT No.: PCT/IT07/00382

§ 371 Date: Nov. 21, 2008

(30) Foreign Application Priority Data
May 31, 2006 (IT) RM2006A000289

(51) Int. Cl. A61F 2/28; A61K 35/12; A61P 17/00; A61P 19/00; A61K 35/28

(52) U.S. Cl. 424/424; 424/93.7

(57) ABSTRACT

A bio-membrane with angiogenic activity for implant in tissue regeneration and repair, including bone reconstruction and the repair of skin and soft tissue lesions is described, essentially constituted by a gel able to provide support and growth and/or differentiation and/or angiogenic factors for the full in vivo functionality of the cell, containing also mesenchymal stem/precursor cells, an implant device for reconstructive surgery of bone tissue, of skin and soft tissue lesions which comprises the bio-membrane, and a method for its obtainment. Use of the gel alone for tissue regeneration and of adhesive plasters that comprise it is also described.

### TECHNICAL FIELD OF THE INVENTION

The present invention relates to an engineered, bio-membrane, an implant device for tissue regeneration and repair as bone reconstruction, repair of lesions of the skin and of soft tissues, e.g. chronic ulcers, difficult wounds, bedsores, chinks, tendon lacerations, soft tissue substance loss, and methods for the production thereof.

### BACKGROUND ART

In clinical practice and in surgery, it is ever more needed to identify a valid system to repair large tissue lesions associated with substance losses.

In orthopedics and maxillofacial surgery, in the past few years a bio-technological approach has been proposed which suggests the use of the patient's own cells in association with ceramic scaffolds, appropriately designed with respect to the lesion to repair large substance losses (Quarto et al, 2001). The results presented in the literature, which are certainly valid, can nonetheless be further improved. Pre-clinical studies, and a pilot clinical study have highlighted that the lack of vascularization at the level of the implant itself can lead to cell death (apoptosis), nullifying the real effect of “bone marrow stromal cells”, BMSC.

In a recent trial in sheep (Mastrogiacomo et al, 2006), a re-absorbable ceramic scaffold was used, and it was observed that, starting from periosteum residues, a bone formation process can be triggered, able to fully repair the lesion. The progressive formation of bone tissue and the filling of the ceramic pores are accompanied by a re-absorption of the ceramic and by the simultaneous vascularization of the implant. Unfortunately, the use of the patient's periosteum is only rarely applicable in reconstructive surgery.

With regard to repair/regeneration of other tissues, the use of techniques like surgical debridement or transplants of vascularized tissue to repair skin lesions due to burns, difficult or post-operation wounds, chronic ulcers, are only one of the few possible applications (Warnke, 2004). Considering the regulatory role in tissue repair that is played by platelets and macrophages, recently the use of local applications of platelet gel has been proposed to repair skin lesions of various kinds (Scala, 2000).

### DESCRIPTION OF THE INVENTION

The authors have now devised a bio-membrane that solves the problems of the prior art and that is able to induce in vivo, in animals and in humans, the production of neo-tissue. Said neo-tissue can be bone, when the bio-membrane is implanted as envelope of a scaffold, such as reabsorbable porous ceramic scaffolds for the repair of large size bone deficits. The neo-tissue can also be a soft tissue as in the repair of skin lesions by direct contact.

Therefore, an object of the present invention is a bio-membrane essentially constituted by mesenchymal stem cells and/or mesenchymal precursor cells and by a gel able to provide support and growth factors and/or differentiation factors, and/or angiogenic factors for the full in vivo functionality of the cells, in which said mesenchymal cells grow within or above said gel.

Preferably, the mesenchymal stem cells and/or mesenchymal precursor cells are dermogenic cells.

Alternatively, the mesenchymal stem cells and/or mesenchymal precursor cells are chondrogenic cells.

Alternatively, the mesenchymal stem cells and/or mesenchymal precursor cells are osteogenic cells.

In a preferred embodiment, the cells are obtained from human and animal bone marrow.

Alternatively, the cells are obtained from periosteum.

In a preferred embodiment, the bio-membrane is pre-treated in culture with osteogenic factors.

Preferably, the cells are autologous. Alternatively, the cells are allogenic. In a preferred form, the gel is a platelet gel. Alternatively, the gel is essentially constituted by reabsorbable synthetic, natural or recombinant polymers, supplemented with growth and/or differentiation and/or angiogenic factors (recombinant or derived from blood) for the full functionality of the cells tasked with regenerating bone tissue and skin lesions.

In an embodiment, the bio-membrane further comprises micro and/or nanoparticles able to release growth and/or differentiation and/or angiogenic factors. Said factors may derive from a platelet lysate or be synthetic, or natural, or specific recombinant products, such as VEGF and PDGF.

The bio-membrane of the invention is advantageously usable if partially dehydrated before its application.

Another object of the invention is an implant device for reconstructive surgery of bone tissue, essentially constituted by a porous support (scaffold) and by the bio-membrane according to the invention, in which the bio-membrane envelops the support and it is preferably pre-treated in culture with osteogenic factors, for a variable time period, such as 1-2 weeks. In an embodiment, the micro and/or nano-particles with gradual release of growth factors can be associated to the porous support.

In a preferred embodiment, the implant device for the reconstructive surgery of bone tissue according to the invention comprises an additional gel membrane with growth and/or differentiation and/or angiogenic factors, in which said additional gel membrane is enveloped just before implanting. Preferably, said additional membrane is a platelet gel. Another object of the invention is the use of a platelet gel for the preparation of a medication for the repair of skin and soft tissue lesions, preferably comprising chronic ulcers, difficult wounds, bedsores, chinks, tendon lacerations, soft tissue substance loss. For the repair of skin lesions, the invention proposes an adhesive plaster that includes only platelet gel. The adhesive plaster is constituted by three essential elements: the pad, the support and the adhesive. The pad can be constituted by cotton mixed with acrylic with high absorption capacity or by a material with similar characteristics and covered by a thin film of polyester or by a material with similar characteristics, loaded with platelet gel rich in active biological factors which, in contact with the wound, accelerates healing.

A further object of the invention is an adhesive plaster for the repair of skin and soft tissue lesions comprising a platelet gel as a therapeutically active substance.

Alternatively, the adhesive plaster for the repair of skin and soft tissue lesions comprises as a therapeutically active substance a gel constituted essentially by reabsorbable synthetic, natural or recombinant polymers supplemented with growth and/or differentiation and/or angiogenic factors.

Alternatively, the adhesive plaster for the repair of skin and soft tissue lesions comprises as a therapeutically active substance micro and/or nanoparticles able to release growth and/or differentiation and/or angiogenic factors.

A further object of the invention is a method for obtaining a bio-membrane according to the invention, essentially comprising the following steps:

a) obtaining a platelet gel from mixing a platelet concentrate and a cryoprecipitate obtained from peripheral blood, in appropriate conditions;

b) obtaining and cultivating said gel or within said gel mesenchymal stem cells and/or mesenchymal precursor cells, from bone marrow (BMSC or stromal cells) or from other tissue.

Preferably, the mesenchymal stem cells and/or mesenchymal precursor cells are autologous or allogenic with respect to the subject to be implanted.

The present invention will now be described in its non limiting examples, referring to the following figures:

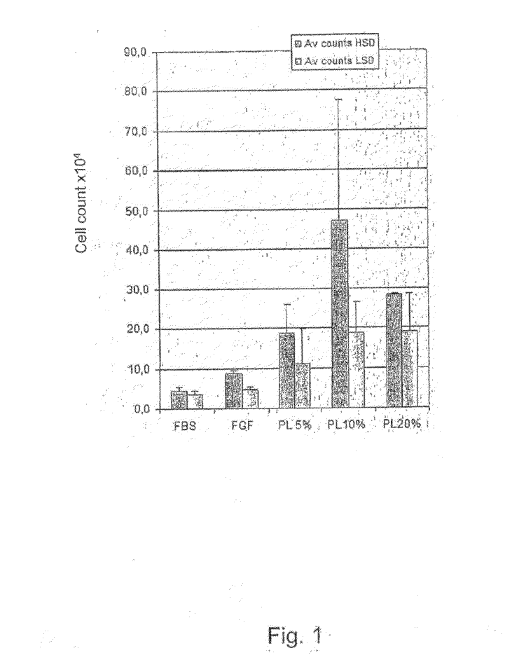

FIG. 1. Histogram of the cell proliferation of human BMSC in the presence of Platelet Lysate (PL) (5%, 10%, 20%), FBS 10% or FGF2 1 ng/ml. Proliferation was evaluated by cell count of wells plated at low cell density (LSD, Low seeding density) and high cell density (HDSD, high seeding density).

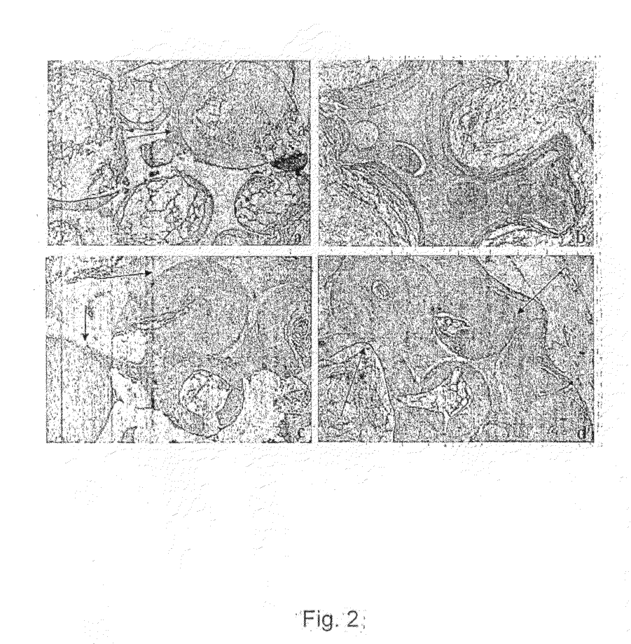

FIG. 2. Bone tissue formation. A film of platelet gel associated with sheep BMSC was wrapped around a cube of hydroxyapatite (HA, 100% pure HA—60-70 mm³) and implanted subcutaneously in immunodeficient mice for 4 and 8 weeks: the cells were bridled within the matrix of the gel (IN) (panels a and c) or layered on the surface of the gel (ON) (panels b and d). Bone tissue formation is highlighted by the hematoxylin-eosin staining indicated by the arrows.

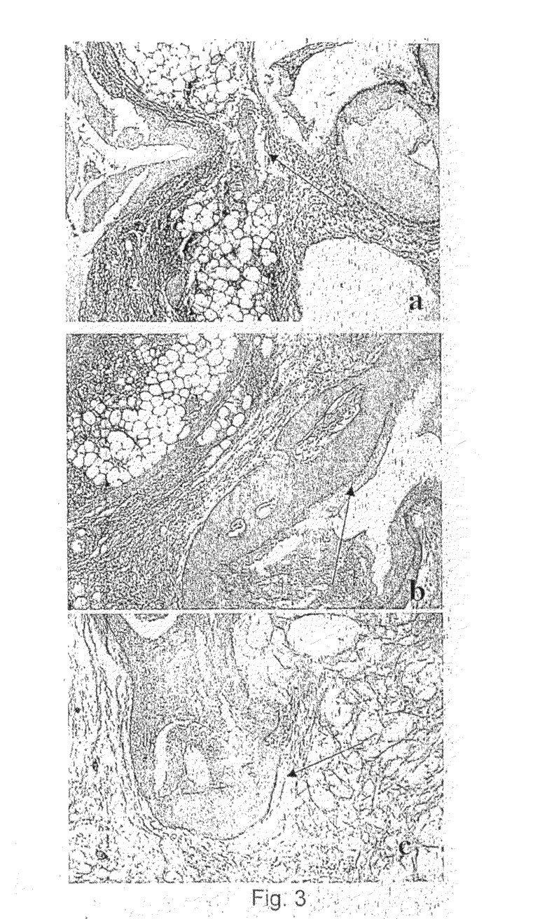

FIG. 3. Bone tissue formation. A film of platelet gel alone (a) or associated with sheep BMSC IN (b) or ON (c) was wrapped around skelite® (TCP-HA—2000-2500 mm³) scaffolds and implanted in immunodeficient mice for 8 weeks. Bone tissue formation is highlighted by the hematoxylin-eosin staining indicated by the arrows.

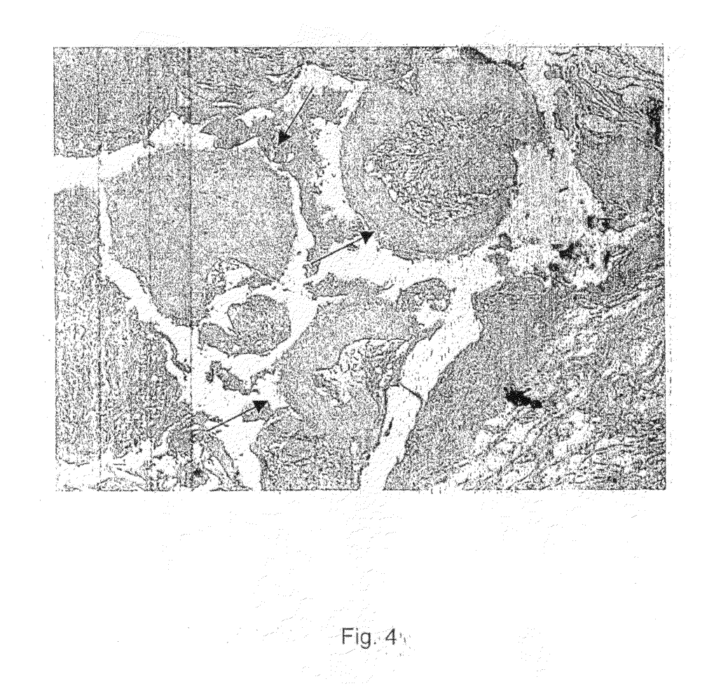

FIG. 4. Bone tissue formation. A film of platelet gel with sheep BMSC on cubic scaffolds (100% pure −64 mm³). The BMSCs were layered on the surface of the platelet gel and stimulated with osteogenic medium for two weeks. Hematoxylin-eosin staining highlights bone tissue formation in the ceramic pores, as indicated by the arrows.

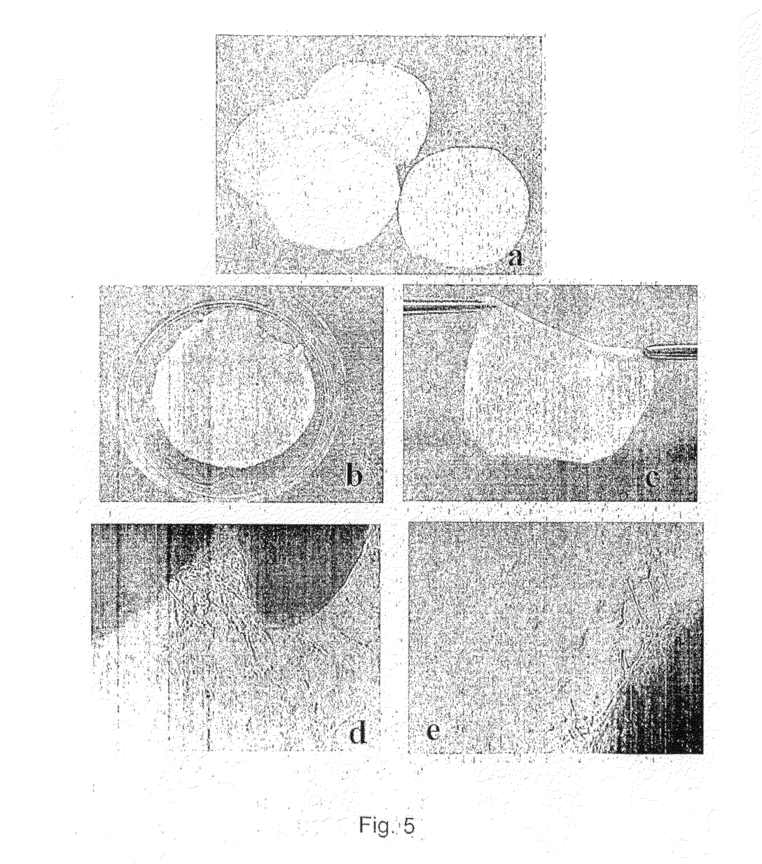

FIG. 5. Dehydration of the bio-membrane. The bio-membrane is dehydrated by means of sterile absorbent paper (a) assuming a consistency and elasticity that enable easily to transpose it into the implant site (b-c). Cell vitality tests demonstrate that the vitality of the cells included in the bio-membrane after dehydration (e) is equal to that of the non dehydrated control.

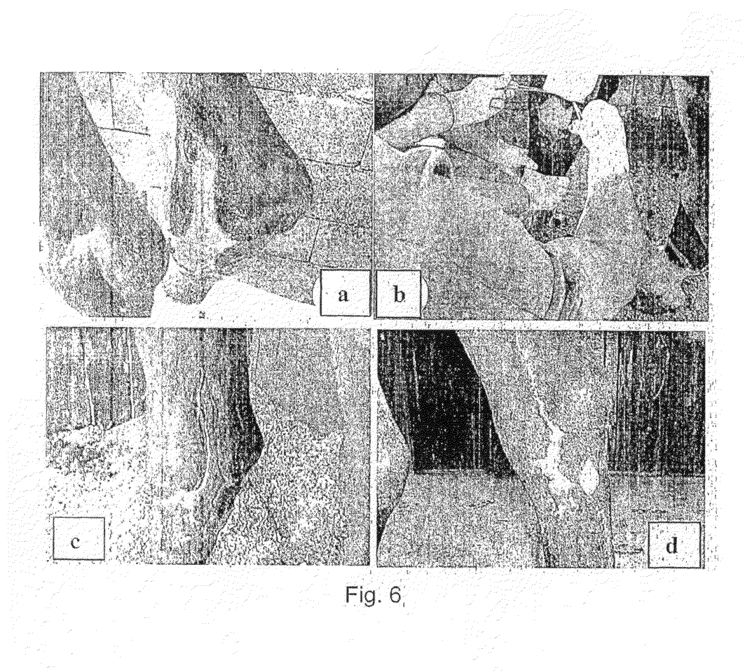

FIG. 6. Repair of a skin lesion in a horse. A bio-membrane constituted by autologous horse platelet gel and hyaluronic acid patch was layered on the lesion.

Cell Cultures

Bone Marrow Stromal Cells (BMSC) were isolated from human or sheep bone marrow. The samples, after authorization by the patient or by the ethical board in the case of trials on animals, were drawn from the iliac crest (10 ml).

In some experiments, cells were derived directly from human or sheep periosteum biopsies by successive digestions with 0.25% of Collagenase according to standard protocols. The bone marrow was washed in PBS and the nucleate cell count per ml of sample was performed. Part of the sample was plated at very low density (100 ml/plate) to evaluate the number of CFU in F12 medium supplemented with 2 mM glutamine, 100 U/ml penicillin and 100 μg/ml streptomycin, 1 ng/ml FGF-2 and 10% of bovine fetal serum. The remaining part of the marrow aspirate was destined to the expansion of the cells in culture in standard culture medium. When the cells reached the first confluence, they were trypsinized and plated on Petri dishes or on platelet gel in the surface (method called IN), or associated to the platelet gel during its polymerization (method called ON). The concentration of the plated cells in the IN or ON gel varies from 1×10⁶ to 6×10⁶ cells per cm² of surface area.

Preparation of Human Platelet Gel

The human platelet gel was obtained from blood components prepared by the Transfusion Center of the San Martino Hospital in Genoa. From the withdrawal of peripheral blood of the human or sheep donor, the following are obtained:

a) a cryoprecipitate containing coagulation factors and immunoglobulins;

b) a platelet concentrate (CP) containing platelets;

c) autologous thrombin that intervenes in the polymerization process of fibrin.

The preparation of the individual components proceeds as follows:

The blood is centrifuged for 7 minutes at 20° C/ at 1700 g/min and allows the separation of a platelet rich plasma called PRP.

The PRP is centrifuged at 4400 g/min for 5 minutes at 20° C. allowing the separation of the platelet poor plasma called PPP and platelet concentrate (CP). The CP is frozen and thawed to ambient temperature at the time of use.

The PPP is frozen at −40° C. and thawed at 4° C. throughout the night in satellite sack. When thawing is complete, the cryoprecipitate is obtained by siphoning.

The CP and the cryoprecipitate were mixed in plate in a 1:1 ratio, 1 ml of autologous thrombin and 1 ml of 10% calcium gluconate on a total volume of 10 ml were added to initiate the gel polymerization process.

To assess the effect of the platelet gel on human BMSC, the cells were grown in the presence of culture medium complete with supplements and with different concentrations of Platelet Lysate (LP) (5%, 10% and 20%), obtained from the CP, as described below. The cells were plated in wells at high density (10,000 cells/well) and at low concentration (2,000 cells/well) in the presence or absence of LP. Cell proliferation was evaluated, in the different conditions, by cell count when the culture had reached semiconfluence (10 days). For the preparation of the Platelet Lysate, the protocol described by Doucet C et al. (2005) was followed. The LP is obtained after subjecting the CP to 3 freezing/thawing cycles to promote complete platelet lysis and total release of all growth factors contained therein (PDGF-bb, PDGF-aa, EGF, IGF etc . . . ) and in presence of low EDTA concentration. The LP was added to the culture at different concentrations.

Preparation of Platelet Gel from an Animal (Horse)

The day before the intervention, two units of 450 ml of blood are drawn from the horse, by means of a standard triple bag for the withdrawal of human blood containing ACD ((citric acid+sodium citrate+dextrose) as an anticoagulant (Fresenius HemoCare CODE T2375). The bags were centrifuged in an ALC PM980R centrifuge (BICase, Italy) for 8 minutes at 500×g; the blood is then separated into red cells and Platelet Rich Plasma (PRP), partially entering into the Buffy-Coat®.

The PRP must be re-centrifuged at 5,000×g for 7 minutes to obtain the Platelet Concentrate (CP) that must be re-suspended in about 80 mL of autologous plasma adjusting platelet count between 0.5 and 3×10⁶ microliter.

The bag containing the CP is placed in an agitator thermostated at +22° until the time of use.

The day of the intervention, the CP is drawn under sterile hood from the bags, with syringes labeled with the identifying data of the horse.

The CP is Ready to be Injected Into the Site of the Lesion to be Repaired

If the product is to be used in the form of semi-solid gel, at the time of use some sterile plastic Petri dishes of about 10 cm diameter are prepared adding 10 mL of platelet concentrate, 1 mL of Calcium Gluconate (ind. Farmaceutica Senese, Italy, Lot. No.

After a few minutes, the transformation occurs from fibrinogen to fibrin with “gelification” of the Platelet Concentrate which can be applied topically on large surfaces.

In case of tendon lesions, the product will be injected non gelified into the site of the lesion, under echographic guidance.

Bone Tissue Formation in Vivo

To evaluate bone tissue formation in vivo, a small animal model was used, i.e. the immunodeficient mouse (nu/Nu strain or SCID strain). Ceramic scaffolds of different sizes and breakdown (Engipore®, 100% HA, Finceramica, Faenza, Italy and Skelite®, TCP70/HA30, Millenium Biologix) were implanted subcutaneously into the back of immunodeficient mice after enveloping them with a bio-membrane of platelet gel and human or sheep BMSC.

The BMSC were layered on the gel (ON) or included in the gel (IN) directly during the polymerization phase. The bio-membrane of platelet gel (obtained with the ON method or with the IN method) was kept in complete medium but without FGF-2 for 1-3 days before being enveloped around cubic scaffolds (60-70 mm³) of HA 100% (EngiPore®),. In some experiments, a few minutes before the implant, the sample was enveloped by an additional membrane of fresh platelet gel without cells, to assure a greater supply of growth factors. In each animal, 4 scaffolds were implanted including a control implant, in which the BMSC were loaded directly into the scaffold using fibrin glue (Tissucol200 , Baxter) as an adjuvant of the adhesion of the cells to the ceramic.

In a second series of experiments, larger size (hollow cylinders of 2000-2500 mm³) reabsorbable ceramic scaffolds made of skelite® (TCP 70%, HA 30%, Millenium Biologix, Ontario, Canada) were used. In this case, a single sample was implanted per animal.

In some experiments, the platelet gel conjugated to BMSC was partially dehydrated by superposing absorbent, sterile filter paper, thereby forming a more consistent and more easily handled bio-membrane. In the partially dehydrated gel, the cells proliferated normally, maintaining their osteogenic potential after implant in the animal.

In some experiments, the platelet gel conjugated to human or sheep BMSC was pre-treated in vitro with osteogenic medium. 24 hours after preparation, the platelet gel membranes were transferred in culture medium supplemented with factors inducing osteogenic differentiation: 10⁻⁸ M dexamethasone, 10 mM b-glycerol-phosphate (BGP), and 50 mg/ml enveloped around HA cubes, re-enveloped by fresh platelet gel without BMSC and implanted subcutaneously in ID mice.

In vivo implants were retrieved after 4 and/or 8 weeks and subjected to histological analysis: the samples were decalcified and enclosed in paraffin. The sections were Hematoxylin-Eosin stained according to standard procedures.

Skin Lesion Repair (Horse)

The bio-membrane, constituted by platelet gel prepared as indicated above, was layered on the lesion and covered by a patch of hyaluronic acid (ComvaTec Hyalofill, FAB Srl, Abano Terme, Italy) or by other material with coverage, characteristics such as OpSite Flexigrid (Smith and Nephew). To the biological implant was then applied a modestly compressive traditional medication with gauze and bandages replaced after 14 days. The follow up was conducted by clinical monitoring of the patient and measuring the surface area of the remaining lesion at regular time intervals after the start of the treatment.

### RESULTS

Proliferation

In the first phase of the work, the effect of the platelet gel on human BMSC was assessed, growing the cells in the presence of culture medium supplemented with serum only, FGF-2 only or with 3 different concentrations of LP. The cells were plated in wells at low or high density. The chart shown in FIG. 1 shows that the cells grown at high or low density in the presence of 5% LP proliferate significantly more than cells grown in serum only. Cells grown in the presence of FGF-2 also exhibit less proliferation than those treated with LP. High BMSC proliferation is observed in medium supplemented with 5, 10 or 20% LP. The addition of LP determines a significant increase in proliferation with respect to the conditions with serum only or FGF-2. While the activity peak is obtained with 10%, in subsequent in vitro experiments the LP concentration used was 5% because it is equally efficient.

In Vivo Differentiation

Human or sheep BMSC were loaded at the surface of the gel (ON method) or directly in the gel mesh (IN method) forming a veritable compact film, called bio-membrane, which was used to envelop a small or large ceramic scaffold.

In FIG. 2, platelet gel bio-membranes prepared with BMSC both with the IN method and with the ON method were enveloped around cubes of 100% HA and implanted for 4 and 8 decalcified samples, paraffin-enclosed samples, it was possible to observe that the cells, both enmeshed in the gel (IN, a,c) and kept on the surface of the gel (ON, b,d) are able to differentiate into osteoblasts and to deposit osteogenic matrix into the ceramic pores already during the first four weeks of implant. A significant line of osteoblasts at the edge of the newly laid bone indicates an intense bone matrix laying activity. After 8 weeks of implant, a greater quantity of bone fills the pores of the ceramic. No significant difference was observed in the formation of bone tissue both in the samples enveloped by bio-membranes with layered cells in the surface (IN method) and in those with bio-membranes with cells enmeshed in the fibrin mesh (ON method).

Though the model of the ID mice is one of the most accredited in vivo models to test the osteogenicity of cells and biomaterials, the authors deemed it appropriate to repeat the experiment under test conditions that would more closely approach the “real” conditions to be found in clinical practice.

For this purpose, a porous, reabsorbable ceramic scaffold was used (Mastrogiacomo et al., 2006), with a greater presence of Tricalcium phosphate and a smaller presence of hydroxyapatite (TCP 70%, HA 30%). Hollow cylinders of about 2,000 mm³ were enveloped with platelet gel bio-membranes, alone or associated with cells with the IN method or with the ON method and implanted in ID mice for 8 weeks. Panel a) in FIG. 3 shows no bone tissue formation in samples enveloped by platelet gel without cells. Only fibrous tissue together with fatty tissue populates the pores of the ceramic. However, some vascularization can be observed, confirming the important role played by the platelet gel in the vascularization of the lesion site (Rhee J J, 2004). When the scaffold is wrapped by film of platelet gel associated with cells obtained with the IN method or with the ON method (FIG. 3b-c), bone tissue is observed in the pores of the ceramic. Abundant osteoblasts are distributed at the surface of the laid bone. Upon microscopic observation of the ample colored section, it is possible to note a distribution of newly formed bone tissue that goes from the periphery to the center of the scaffold.

In the attempt to generate a bio-membrane with the highest osteogenic and angiogenic potential starting from a bio-membrane of platelet gel and BMSC (human or sheep) obtained both with IN method and with ON method, the bio-membranes were pre-treated in vitro for a period of 2 weeks with osteoinductive culture medium (see methods), to promote an initial laying of matrix prior to the transfer on scaffold. After 8 weeks of in vivo implant, the samples (FIG. r) exhibited good bone tissue formation distributed from the periphery to the center of the samples.

The prolonged maintenance of BMSC in platelet gel in vitro reduces their osteogenic potential. However, the bio-membranes can be pre-treated with osteoinductive medium for a period of two weeks, assuring the maintenance of the full osteogenic potential.

In FIG. 5 we show the dehydration of the bio-membrane by means of a continuous superposition of disks of sterile absorbent paper that completely removes the soluble part of the membrane and water. This procedure generates a membrane that is more elastic and easier to handle during the surgical procedure without altering the vitality of the cells included therein.

With respect to the repair of skin lesion, an example of treatment of skin lesion in a horse is reported (FIG. 6). In all treated animals, it was sufficient to apply the platelet gel once to trigger the regenerative process (FIG. 6a-b). The figure clearly shows the reduction of the lesion at 15 days from the treatment (FIG. 6c) and restitution ad integrum after thirty days (FIG. 6d) when the horse resume its sports-competition activity.

### BIBLIOGRAPHY

1) Quarto et al., N. Engl. J. Med., 2002, 344 (5); 385-86.

2) Mastrogiacomo et al., Tissue Eng. 2006, 12(5): 1261-73.

3) Rhee et al., Thromb Haemost. 2004, 92(2): 394-402.

4) Scala M et al., Proc. Am Acad Maxillofacial Prosth. International Congress Maxillofacial Prosthetics in the 21^{st} Century, Kauai-Hawaii; Nov. 11-14, 2000: 132.

5) Warnke P H et al., Lancet 2004, 364: 766-70.

1. A bio-membrane essentially constituted by mesenchymal stem cells and/or mesenchymal precursor cells and by a gel able to provide support and growth factors and/or differentiation factors and/or angiogenic factors for the full in vivo functionality of the cells, in which said mesenchymal cells grow within or above said gel.

2. The bio-membrane as claimed in claim 1, wherein the mesenchymal stem cells and/or mesenchymal precursor cells are dermogenic cells.

3. The bio-membrane as claimed in claim 1, wherein the mesenchymal stem cells and/or mesenchymal precursor cells are chondrogenic cells.

4. The bio-membrane as claimed in claim 1, wherein the mesenchymal stem cells and/or mesenchymal precursor cells are osteogenic cells.

5. The bio-membrane as claimed in claims 1 through 5 wherein the cells are obtained from bone marrow.

6. The bio-membrane as claimed in claim 4, wherein the cells are obtained from periosteum.

7. The bio-membrane as claimed in claim 4, wherein the bio-membrane is pre-treated in culture medium with osteogenic factors.

8. The bio-membrane as claimed in one of the previous claims wherein the cells are autologous.

9. The bio-membrane as claimed in one of the claims from 1 through 7 wherein the cells are allogeneic.

10. The bio-membrane as claimed in one of the previous claims wherein the gel is a platelet gel.

11. The bio-membrane as claimed in one of the claims 1 through 9 wherein the gel is essentially constituted by reabsorbable synthetic, natural or recombinant polymers, supplemented with growth and/or differentiation and/or angiogenic factors for the full functionality of the cells.

12. The bio-membrane as claimed in one of the previous claims, further comprising micro and/or nanoparticles able to release growth factors and/or differentiation factors and/or angiogenic factors.

13. The bio-membrane as claimed in one of the previous claims being partially dehydrated.

14. An implant device for reconstructive surgery of bone tissue, essentially constituted by a porous scaffold and by the bio-membrane as claimed in claim 4 or 6, wherein the bio-membrane envelops the support.

15. The implant device for reconstructive surgery of bone tissue as claimed in claim 14, wherein the bio-membrane is pre-treated in culture medium with osteogenic factors.

16. The implant device for reconstructive surgery of bone tissue as claimed in claim 14 or 15, further comprising an additional gel membrane with growth and/or differentiation and/or angiogenic factors, wherein said additional gel membrane is enveloped shortly before implanting.

17. The implant device for reconstructive surgery of bone tissue as claimed in claim 16, wherein said additional gel membrane is a platelet gel.

18. Use of a platelet gel for the preparation of a medication for repairing skin and soft tissue lesions.

19. Use of a platelet gel as claimed in claim 18, wherein the repair of skin and soft tissue lesions comprises chronic ulcers, difficult wounds, bedsores, chinks, tendon lacerations, soft tissue substance loss.

20. An adhesive plaster for the repair of skin and soft tissue lesions comprising a platelet gel as claimed in claim 18 or 19 as a therapeutically active substance.

21. An adhesive plaster for the repair of skin and soft tissue lesions comprising as a therapeutically active substance a gel constituted essentially by reabsorbable synthetic, natural or recombinant polymers supplemented with growth and/or differentiation and/or angiogenic factors.

22. An adhesive plaster for the repair of skin and soft tissue lesions comprising as a therapeutically active substance micro and/or nanoparticles able to release growth and/or differentiation and/or angiogenic factors.

23. A method for obtaining a bio-membrane as claimed in one of the claims 1 through 13, essentially comprising the following steps:
a) obtaining a platelet gel from mixing a platelet concentrate and a cryoprecipitate obtained from peripheral blood, in appropriate conditions;
b) obtaining a growing on said gel, or within said gel, mesenchymal stem cells and/or mesenchymal precursor cells.

24. The method for obtaining a bio-membrane as claimed in claim 23 wherein the mesenchymal stem cells and/or mesenchymal precursor cells are autologous or allogeneic with respect to the subject to be implanted.
